(19) United States
(12) Patent Application Publication
(10) Pub. No.: US 2019/0328985 A9
(43) Pub. Date: Oct. 31, 2019

(54) INHALATION DEVICE WITH CONSUMPTION METERING INCLUDING ONE OR MORE AIRFLOW SENSORS

(71) Applicant: INDOSE INC, Woodland Hills, CA (US)

(72) Inventors: Daniel Freeman, Agoura, CA (US); Ari Freeman, Lafayette, CA (US); Jacqueline Freeman, Lafayette, CA (US)

(73) Assignee: INDOSE INC., Woodland Hills, CA (US)

(21) Appl. No.: 15/990,924

(22) Filed: May 29, 2018

Related U.S. Application Data
(63) Continuation-in-part of application No. 15/244,518, filed on Aug. 23, 2016.
(60) Provisional application No. 62/621,795, filed on Jan. 25, 2018.
(60) Provisional application No. 62/386,615, filed on Dec. 7, 2015.
(60) Provisional application No. 62/388,066, filed on Jan. 13, 2016.
(60) Provisional application No. 62/386,614, filed on Dec. 7, 2015.

(51) Int. Cl. A61M 15/00; A24F 47/00; A61M 15/06

(52) U.S. Cl. CPC A61M 15/0065; A24F 47/008; A61M 11/042; A61M 15/06; A61M 15/0086

(57) ABSTRACT

An inhalation device for inhaling a vaporized substance that includes metering capabilities to inform a user when a particular amount of substance has been consumed. The inhalation device can include an inlet, an outlet, a channel positioned between the inlet and outlet. The device can further include an atomizer positioned between the inlet and the outlet and configured to vaporize an unvaporized substance into a vaporized substance, where the vaporized substance flows downstream from the atomizer to the outlet via the channel. The inhalation device can further include an airflow sensor positioned upstream of the flow of the vaporized substance, where the airflow sensor is configured to acquire information on the flow of air from the inlet.

This application is a continuation-in-part of and claims priority to U.S. patent application Ser. No. 15/244,518, filed on Aug. 23, 2016, which in turn claims priority to U.S. Provisional Patent Application Nos. 62/386,614 and 62/386,615, both of which were filed on Dec. 7, 2015, and 62/388,066, which was filed on Jan. 13, 2016. This application also claim priority to U.S. Provisional Patent Application No. 62/621,795 filed on Jan. 25, 2018. All of these applications are incorporated by reference herein in their entireties.

### BACKGROUND

Inhaling devices such as vaporizers, vaporizing pens, and vaporizing machines are used to vaporize substances such as tobaccos, oils, liquids, medical drugs, and plant herbs. Once vaporized, these substances are then inhaled by consumers. Such inhaling devices have health benefits over traditional smoking methods. But inhaling the vapor can have negative effects on the body depending on the substance, such as nicotine. Inhaling devices have become more popular with consumers, but pose problems.

For example, while vaporizers can be safer than traditional smoking methods, it is difficult to meter the amount of vaporized substance that is being inhaled. So a user of an inhalation device that vaporizes nicotine may actually consume more nicotine than had the user smoked cigarettes or cigars.

There are multiple factors that affect the quantity of drug that is inhaled. These factors include the drug concentration of the vaporized substance, the amount of vapor inhaled, the duration of inhalation, variations between inhalation devices, and variation and inconsistency in the functionality of the device.

Another issue is that the inhaled substances may have different effects on different users depending on various factors. To optimize a user's experience, it is necessary to track the quantity inhaled taken over time and track the resulting effect it has on that user. This can be a tedious and demanding task. Typical users may not keep track of each dose and record the experience.

### SUMMARY

Various aspects and embodiments of inhalation devices are provided in this disclosure. In one aspect, this disclosure describes an inhalation device that includes metering capabilities to inform a user when a particular amount of substance has been consumed. The inhalation device can include an inlet, an outlet, a channel positioned between the inlet and outlet. The device can further include an atomizer positioned between the inlet and the outlet and configured to vaporize an unvaporized substance into a vaporized substance, where the vaporized substance flows downstream from the atomizer to the outlet via the channel. The inhalation device can further include an airflow sensor positioned upstream of the flow of the vaporized substance, where the airflow sensor is configured to acquire information on the flow of air from the inlet.

In another aspect, the disclosure provides an inhalation device for inhaling a vaporized substance including an inlet, an outlet, a channel positioned between the inlet and outlet. The disclosure further provides an atomizer positioned between the inlet and the outlet and configured to vaporize an unvaporized substance into a vaporized substance, wherein the vaporized substance flows downstream from the atomizer to the outlet via the channel, an airflow sensor positioned upstream of the flow of the vaporized substance, wherein the airflow sensor is configured to acquire information on the flow of air from the inlet.

### DESCRIPTION OF THE DRAWINGS

FIG. 1 is a diagram of an inhalation device.

FIG. 1A is a diagram of a portion of an inhalation device.

FIG. 1B is another diagram of a portion of an inhalation device.

FIG. 2 is another diagram of an inhalation device, according to an embodiment of this disclosure.

FIG. 3 is another diagram of an inhalation device, according to an embodiment of this disclosure.

FIG. 4 is another diagram of an inhalation device, according to an embodiment of this disclosure.

FIG. 5 is another diagram of an inhalation device, according to an embodiment of this disclosure.

FIG. 6 is another diagram of an inhalation device, according to an embodiment of this disclosure.

FIG. 7 is another diagram of an inhalation device, according to an embodiment of this disclosure.

FIG. 8 is another diagram of an inhalation device, according to an embodiment of this disclosure.

FIG. 9 is another diagram of an inhalation device, according to an embodiment of this disclosure.

FIG. 10 shows a graph of the value percent drop in an optocell (i.e., a device that senses the intensity of light) versus the percentage of vaporized drug in a mixture of vapor and air.

### DETAILED DESCRIPTION

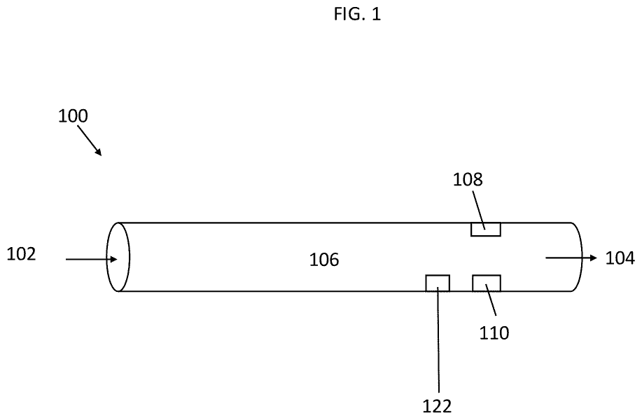

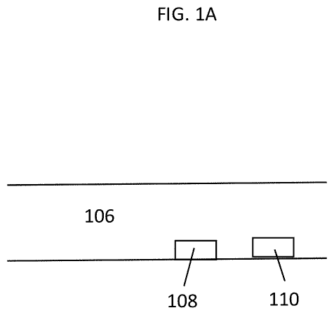

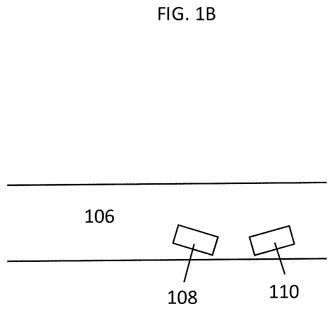

FIG. 1 illustrates an inhalation device 100 for inhaling a vaporized substance. The inhalation device 100 includes a first opening 102 and a second opening 104. In between the two openings is a channel 106. When a user inhales using the inhalation device 100, air flows into the first opening 102 and in the device 100, vaporized substance is created by a heating element (not shown), and a mixture of air and vapor flows through the channel 106 to the second opening 104 and ultimately to the user.

The inhalation device 100 also includes a sensor 108, a signal 110, and an airflow sensor 122. The sensor 108 and signal 110 are positioned across from each other in the channel 106. The sensor 108 senses the vapor amount. For example, the sensor 108 can sense the concentration of vapor. The sensor 108 senses the intensity of the signal emitted by the signal 110. If the sensor 108 senses a high signal output, this indicates that the amount of vapor is low, and the vapor/air mixture is dominated by air. Likewise, if the sensor 108 senses a low signal output, this indicates that the vapor/air mixture is dominated by vapor.

Data from the sensor 108 can assist the device 100 in providing information about vapor concentration to the user. For example, if the sensor senses a 5% drop in intensity from the signal 110, that could correlate to a mixture of vapor/air that is 60% vapor.

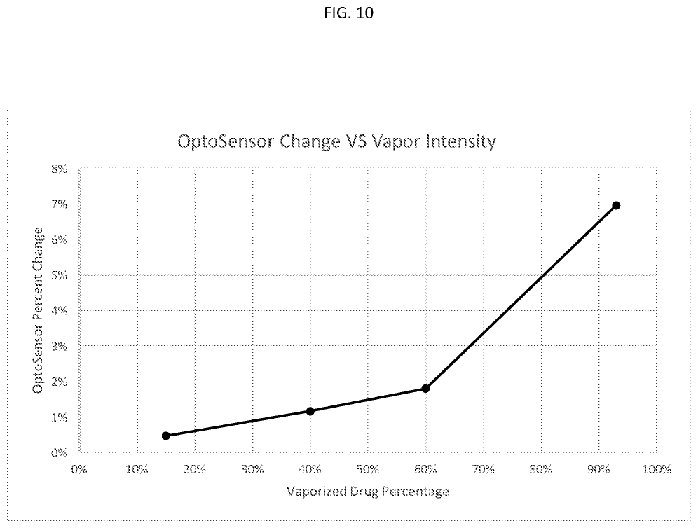

FIG. 10 shows a graph of the value percent drop in an optocell (i.e., a device that senses the intensity of light) versus the percentage of vaporized drug in a mixture of vapor and air.

The chart above shows the correlation between vapor concentration and the readings from an optocell. Knowing the relative concentration of the vapor can assist the device 100 in providing additional information to the user. For example, if a user inhales using the device 100 and the sensor 108 senses a high output, this may indicate that the concentration is less than expected. The device 100 could include an additional indicator to inform the user that the device 100 is not producing the expected amount of vapor. The sensor 108 can be any suitable sensor that senses light including without limitation, a photosensor, photodetector, optocell, optoresistor, optotransistor, optodiode, and/or solar cell. The signal 110 can be any suitable device that produces light, such as an LED. The signal could also emit ultraviolet light. In other words, the signal 110 can produce a wide range of wavelengths of light and the sensor 108 detects those wavelengths of light. The inhalation device 100 can optionally use filters in order to target a specific wavelength of light to optimally detect vapor intensity.

In addition, the signal 110 can also be tuned to particular wavelengths or a plurality of wavelengths to detect specific types of molecules and quantities of these molecules that are present in the passing vapor. This would allow identification and quantification of drugs in vaporized form. This technology can be fitted in a small and limited space such as a compact inhalation device. The vapor itself can remain in its current unaltered state during analysis. The technology allows for real-time analysis as it is being inhaled by the user. Several wavelengths of light may be used concurrently.

This technology can also be used for an exhalation device. In this configuration, we can analyze the air or vapor exhaled by a user. One such use of this configuration is to quantify the amount of drug that is being exhaled after partial absorption in the lungs. Another use of this configuration may be to make a determination on the level of drug within a human by way of analyzing the exhaled air/gas.

In FIG. 1, the sensor 108 is positioned across from the signal 110. The sensor 108 and the signal 110 can also be positioned in alternative arrangements without departing from the scope of this disclosure. For example, in FIG. 1A the sensor 108 and the signal 110 are positioned next to each other in the channel 106. In another embodiment, shown in FIG. 1B, the sensor 108 and the signal 110 are positioned next to each other at an angle in the channel 106. The arrangements of the sensor 108 and the signal 110 in FIGS. 1A and 1B use concepts of backscatter and fluorescence.

In backscatter, the vapor passing through the channel 106 can “reflect” light back from the perspective of the sensor 110. In this scenario, the vapor particle size would determine the “reflection” properties and angle of refection. In florescence, the light may get absorbed by the vapor particles and a new light may be generated. The new light would then be picked up by the sensor. The light and sensor may be set up facing the same direction (in parallel) towards the channel 106. Other alternative positions of sensor 108 and signal 110 known to persons of ordinary skill in the art whereby the flow of vaporized substance affects the signal received by the sensor from the light produced by the light signal device is intended to fall within the scope of this disclosure. For example, the sensor 108 and the signal 110 may be next to each other but one of the sensor 108 and the signal 110 may also be positioned at an angle.

The inhalation device 100 further includes an airflow sensor 122. The airflow sensor 122 can be any suitable airflow sensor including, but not limited to, any combination or stand-alone of the following: an air flow sensor, a propeller, a microphone or a piezoelectric sensor. The airflow sensor 122 is used to measure the velocity at which the mixture of vapor and air flow through the channel 106. So for example, if the sensor 122 is a propeller, the propeller would be installed in the channel 122 and would spin according to velocity of the vapor/air mixture. The frequency of revolutions can be measured and used to calculate the velocity of the mixture. If the sensor is a microphone, the microphone can be setup in the channel 106 to listen to the noise of the vapor/air mixture passing through the channel. A correlation can be made between the sound intensity and/or frequency to the rate of flow of the mixture.

The airflow sensor 122 can be used with the sensor 108 and the signal 110 to meter the amount of vaporized substance that is consumed by a user. For example, the sensor 108 and signal 110 can be employed, as described above, to determine the concentration of the vapor, and the airflow sensor 122 can sense the velocity of the vapor/air mixture. As will be appreciated by persons having ordinary skill in the art, this data can be used to meter the quantity of vaporized substance the user inhales. For example, by experimentation using different airflow rates and vapor concentrations, data can be accumulated from which a predicting formula can be determined. This formula can use airflow data which is converted to a factor and vapor data that is converted to a factor to determine amounts of vapor consumed.

In the embodiment of FIG. 1, the airflow sensor 122 is positioned proximately to the sensor 108 and the signal 110. In this embodiment, the airflow sensor is downstream of the heating element (not shown) and thus the vapor/air mixture will pass over airflow sensor 122, as a user inhales. A potential issue is that the airflow sensor 122 over time will become contaminated by the flow of vapor wherein vaporized substance may settle on the airflow sensor 122.

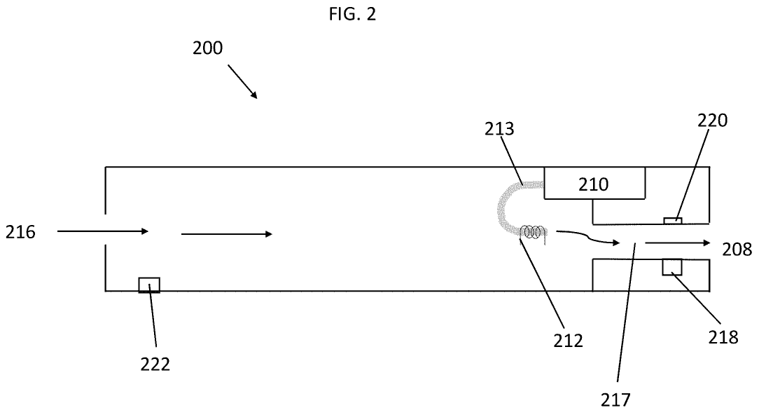

To account for this possibility, in another embodiment, shown in FIG. 2, an airflow sensor 222 in an inhalation device 200, can be positioned substantially away from the flow of vapor. More specifically, the inhalation device 200 includes an inlet 216, an outlet 208, a reservoir 210, a heating element 212, and a wick 213. The inhalation device 200 also includes a signal 218 and a sensor 220. The reservoir 210 stores the substance in unvaporized form, and the heating element 212 heats the unvaporized substance from the reservoir 210 via the wick 213 to create a vaporized substance, which is then inhaled by the user through the outlet 208. The device 200 also includes a channel 217 through which the vaporized substance produced by the heating element 212 and air will flow to the outlet 208 when a user inhales.

While the embodiment of FIG. 2 includes a wick and heating element, other suitable methods of vaporizing a substance could be utilized without departing from the scope of this disclosure. For example, the substance to be vaporized could be placed in a chamber or oven. The oven can be a small cup made of metal, where a user could place the substance. The oven would then heat up and vaporize the substance. Any vapor produced can exit the oven and flow to the user when the user inhales.

The signal 218, can be an LED that produces a wide range of light wavelengths. The signal 218 could also be one that produces ultraviolet light. The sensor 220 and signal 218 are positioned across from each other in the channel 217. The sensor 220 senses the concentration of the vapor. For example, the sensor 220 can be an optical sensor that senses the intensity of the light produced by the signal 218. If the sensor 220 senses a high output, this indicates that the vapor concentration is low, and the vapor/air mixture is mostly, if not all, air. If the sensor 220 senses a low output, this indicates that the vapor concentration is high.

In the embodiment of FIG. 2, the airflow sensor 222 is positioned proximately to the inlet 216, upstream of vapor production. In this embodiment, the vaporized substance may not contact the sensor 222, or any contact will be less than if the sensor 222 is proximate to the outlet 208. In this setup, the chance that vaporized substance contaminates or settles on the sensor 220 is reduced. This is an advantage as contamination of the sensor may cause damage or cause the sensor to report information that is not as accurate.

Positioning this sensor 220 upstream of vapor production, as described above, may yield different pressure/airflow readings than if placed downstream of vapor production. This may be due to the different configuration, different chamber dimensions, different cross sectional dimensions, different density of air/vapor in the space, physical features impeding the flow, temperature changes, different absolute or relative pressures. These variables may make it difficult to derive the air flow within a different area of the air/vapor flow pathway based on a air flow reading from the remote location described above.

These variables, however, can be overcome by applying a specific and known air flow through the system and recording the sensor readings. This process can be repeated for various air flow rates in order to determine the correlation between the sensor readings and actual air flow rates. A correlation can therefore be created between the sensor readings and the flow rate of air thru the unit. This relationship can be saved and used in the future for deriving the flow rates based on the sensor output readings. A further relationship can be derived between the sensor readings and the flow rate of the vapor/air mixture from the outlet in the device.

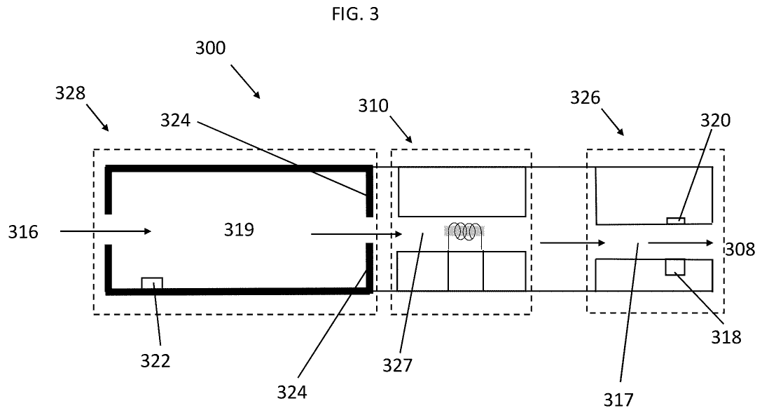

FIG. 3 illustrates another embodiment of an inhalation device 300. In this embodiment, the device 300 includes an inlet 316, an outlet 308, a vapor-creating device, referred to generally as an atomizer 310. The inhalation device 300 also includes a signal 318 and a sensor 320. The atomizer 310 produces vapor that a user inhales through the outlet 308. The signal 318 and sensor 320 are positioned downstream of the atomizer 310 for sensing concentration of the vapor that flows in a channel 317, as described in other embodiments herein. Upstream of the atomizer 310 is an airflow sensor 322, which senses the flow of air that comes in from the inlet 316 (when a user inhales) and flows through a channel 319 which is positioned between the inlet 316 and the atomizer 310. Barriers 324 at the end of the channel 319 can be positioned between the channel 319 and the atomizer 310 to restrict any vapor that may flow upstream towards the sensor 322 when a user stops inhaling. As will be recognized by persons having ordinary skill in the art, when a user stops inhaling the atomizer 310 will still produce vapor as it cools down. Additionally, there is residual vapor that can linger during the cooling down of the atomizer 310. As a result, there will be vapor in the atomizer space, but no airflow (since the user has stopped inhaling), which can cause this additional and residual vapor to move to all areas in the inhalation device 300, including to areas upstream of the atomizer 310, such as where the sensor 322 is located. Thus to minimize movement of vapor upstream, the barriers 324 help restrict the flow of vapor upstream of the atomizer 310. With the barriers 324, the channel 319 can be thought of as a separate space or chamber from the atomizer 310. A person having ordinary skill will also appreciate that any suitable barrier can be used to restrict movement of vapor from the atomizer 310 upstream to channel 319 wherein the sensor 322 is located. Furthermore, while two channels, 317 and 319, have been described, a person of ordinary skill will understand that FIG. 3 illustrates a third channel 327, located between channels 317 and 319 and goes through the atomizer 310. In addition, additional or fewer channels than those described in FIG. 3 can be employed without departing from the scope of this disclosure.

The inhalation device 300 can also be viewed as containing three parts, a chamber 328, the atomizer 310, and a vapor sensing unit 326. The chamber 328 contains the channel 319 wherein the sensor 322 is located. In an alternative embodiment, the chamber 328 could include additional features such as a rechargeable battery, microprocessor, dosage indicator, and puff sensor, without departing from the scope of this disclosure. The vapor sensing unit 326 contains the sensor 320 and the signal 318 to detect the concentration of vapor inhaled by a user. The atomizer 310 is as described above. In FIG. 3, a person of ordinary skill will understand and appreciate that the atomizer 310 can be a separate component to which the chamber 328 can be added upstream of the atomizer 310 and the vapor sensing unit 326 can be added downstream of the atomizer 310.

It should also be understood that the chamber 328, the atomizer 310, and the vapor sensing unit 326 may be detachable from one another. For example, the inhalation device 300 can be used as a cartridge-style device. These types of devices have some portion that is reusable and another portion (i.e., the cartridge) that is disposable. A person of ordinary skill will understand that in some inhalation devices, a cartridge can constitute an atomizer and a substance reservoir. In the inhalation device 300, the cartridge can include a substance reservoir, atomizer 310, and vapor sensing unit 326. The reusable portion would then be the chamber 328 as described above.

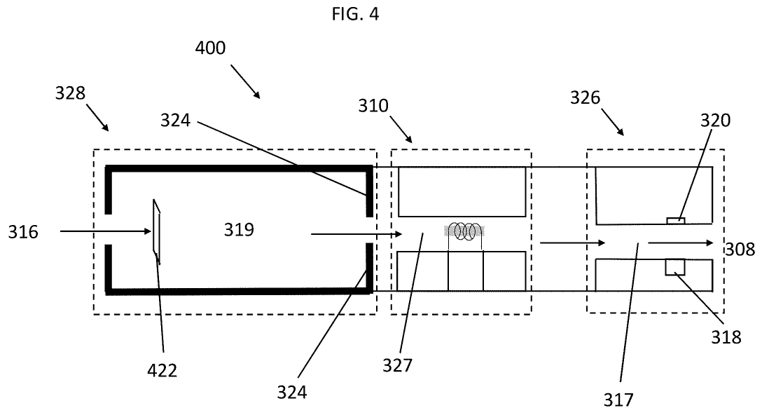

FIG. 4 illustrates another inhalation device 400 according to another aspect of this disclosure. Device 400 is similar to device 300 with the exception that rather than the sensor 322, there is a fin 422 which serves as an airflow sensor to sense the flow of air that comes in from the inlet 316 when a user inhales. More specifically, when a user inhales, air will flow from the inlet 316 into the device 400, and will flow past the fin 422, which will cause the fin 422 to move, either in a particular direction, or can cause the fin 422 to vibrate depending on the kind of fin 422 used, as recognized by persons having ordinary skill in the art. The vibrations or movement may be measured and a corresponding airflow rate determined based on a correlation derived by previous experimentation.

The fin 422 may also be positioned as to bend, turn, compress or stretch. This motion may be measured and a corresponding airflow rate determined based on a correlation derived by previous experimentation. The motion of the fin 422 may be measured by various means such as optic sensors, rotational motion sensors, resistance measurements, piezoelectric sensors and/or capacitance change created by the motion of the fin. Alternatively, the fin 422 may be shaped as a propeller and positioned in the airflow/vapor flow pathway to spin as the air/vapor passes. The speed of rotation may be measured and an airflow speed derived by calculation or by previous experimentation. The fin 422 may be used in conjunction with the sensor 320 and the signal 318 to meter the amount of vaporized substance consumed by the user. Alternatively, the fin 422, as well as any airflow sensor described herein that is positioned upstream of vapor-creation, may be used as a puff detector/switch (to detect the start and stop of a puff).

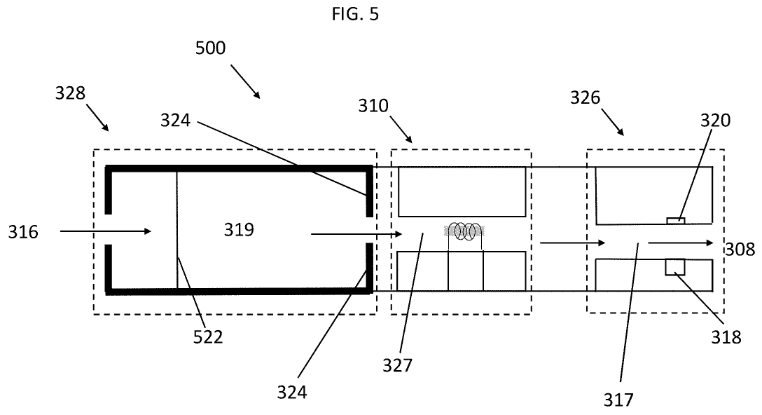

FIG. 5 illustrates an inhalation device 500 according to another embodiment of this disclosure. Device 500 has the attributes of the device 400, except that rather than the fin 422, there is a wire 522 that can be heated and used to detect airflow at the inlet 316. More specifically, the heated wire 522 positioned in the airflow such that the passing air will create a drop in the temperature of the wire. The faster the flow, the more the temperature will drop. The temperature can be measured in real time and a correlating airflow rate may be determined by mathematical calculations or by a look up table. The look up table may be generated beforehand by experimentation of airflow versus temperature in this setup. As will be recognized by persons having ordinary skill in the art, the wire can be made of any suitable conductive material such as copper, steel, or aluminum. As with the fin 422, the heated wire 522 can also be used as a puff detector/switch (to detect the start and stop of a puff) or to measure airflow rates.

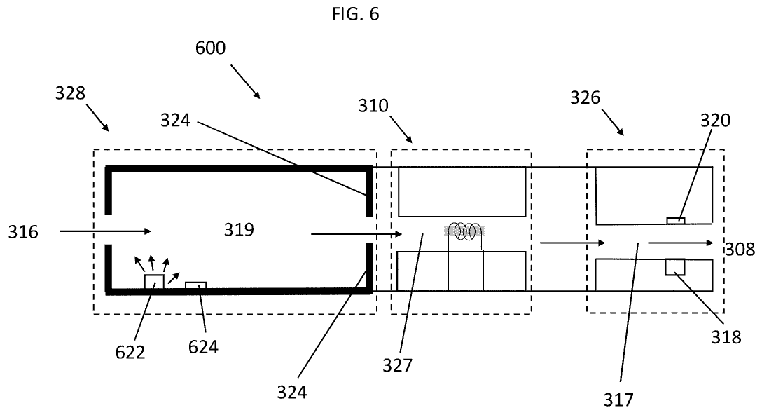

FIG. 6 shows another embodiment of an inhalation device 600 according to another embodiment of this disclosure. Device 600 has the attributes of devices 400 and 500 except that the airflow sensor is a heated element 622 that is located upstream of the atomizer 310. The element 622 may be heated to a specific temperature. A temperature sensor 624 may be located downstream from the heated element 622 in order to measure the temperature of the passing air from the inlet 316. The passing air 316 will be heated by the heating element 622 and then the temperature sensor 624 will measure the temperature of that air. Different air flow rates will result in different temperature readings. The corresponding airflow rate may be determined based on a correlation derived by previous experimentation on the relationship. As with previous embodiments of airflow sensors, the element 622 and temperature sensor 624 may be used as a puff detector/switch (to detect the start and stop of a puff). The heated element 622 can be any suitable material such as a resistor made of metal or ceramic.

In an alternative embodiment, the heated element 622 can be heated by electrical current flowing through the element 622. The passing airflow at the inlet 316 will change the temperature of the element 622. These changes in temperature can create variations in the current drawn by the element 622, and or variations in the resistance across said element.

These variations in current/resistance may be measured. The airflow speed may be derived from these measurements by calculations or by previous experimentation. This embodiment would also include an Amp meter or Ohm meter to measure current or resistance changes.

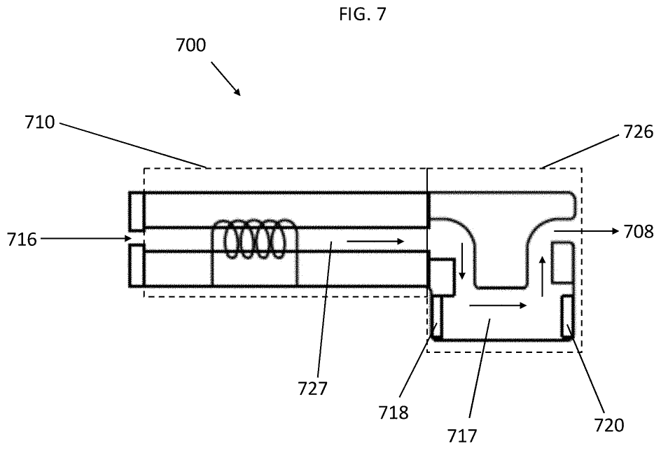

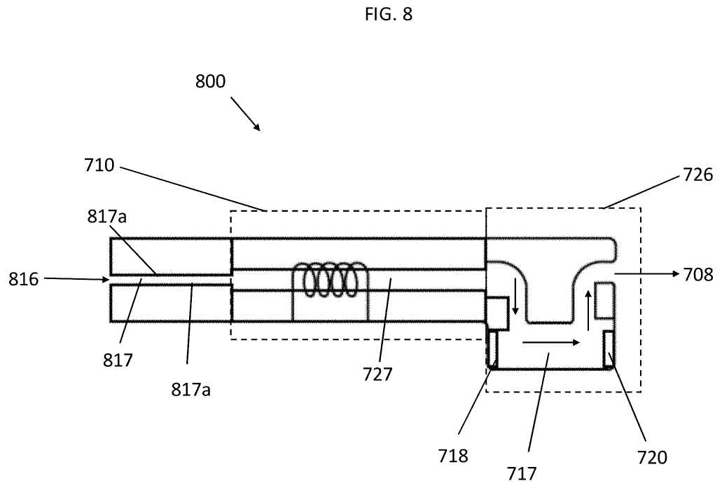

FIG. 7 illustrates an inhalation device 700 according to another embodiment. More specifically, inhalation device 700 includes an inlet 716, an atomizer 710, a vapor sensing unit 726 and an outlet 708. The atomizer 710 includes a channel 727 and the vapor sensing unit 726 includes a signal 718, a sensor 720, and a channel 717. The atomizer 710 produces vapor that a user inhales through the outlet 708. The vapor will flow in the channel 727 of the atomizer 710 and through channel 717 of the vapor sensing unit 726 before flowing through the outlet 708. The signal 718 and sensor 720 are positioned for sensing concentration of the vapor that flows in a channel 717, as described in other embodiments herein. However, a person of ordinary skill in the art will appreciate that the position of the signal 718 and the sensor 720 in FIG. 7 is different than that, for example, in FIG. 3. In FIG. 3, the signal 318 and sensor 320 were above and below the channel 317. In FIG. 7, the signal 718 and sensor 720 are positioned on ends of the channel 717. FIG. 8 illustrates an inhalation device 800 according to another embodiment. FIG. 8 includes the elements of FIG. 7 with the exception being that an inlet 816 of FIG. 8 is longer than the inlet 716 of FIG. 7, and comprises a channel 817. The channel 817 is used to control and limit the air flow rate through this channel by surface tension and friction between the air and the sidewalls 817a of the channel 817.

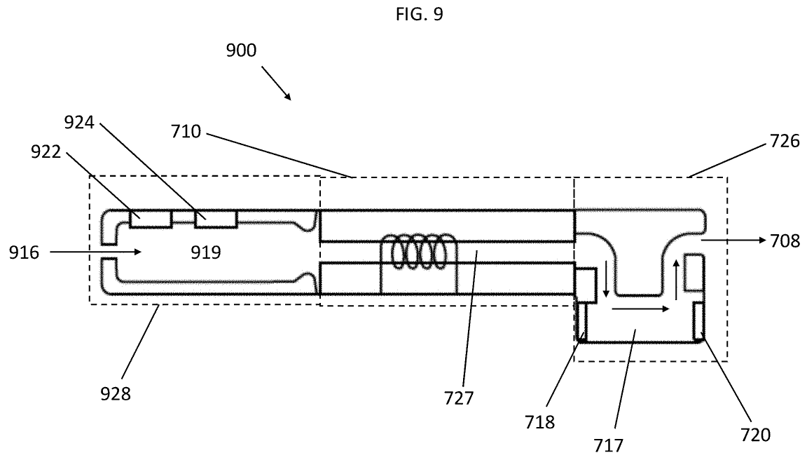

FIG. 9 illustrates an inhalation device 900 according to another embodiment. FIG. 9 includes elements of FIG. 7 but also includes a chamber that includes an air flow sensor 922 and a puff switch 924, as well as a channel 919 in which air flows from the inlet 916 through to the atomizer 710 when a user inhales. The air flow sensor 922 can be those described in previous embodiments, and the puff switch 924 can be used to detect the start and stop of a puff.

While embodiments have been illustrated and described herein, it is appreciated that various substitutions and changes in the described embodiments may be made by those skilled in the art without departing from the spirit of this disclosure. The embodiments described herein are for illustration and not intended to limit the scope of this disclosure.

1. An inhalation device for inhaling a vaporized substance comprising:
an inlet;
an outlet;
an atomizer configured to vaporize an unvaporized substance into a vaporized substance;
a first channel positioned between the inlet and the atomizer;
a second channel positioned between the atomizer and the outlet, wherein the vaporized substance flows downstream from the atomizer to the outlet via the second channel;
a light signal device, wherein the light signal device emits light;
a light sensor, wherein the light sensor senses the light from the light signal device;
an airflow sensor positioned in the first channel;
wherein the light signal device and the sensor are positioned in the second channel such that the vaporized substance can flow past the sensor and the light signal device; and
wherein the airflow sensor is configured to acquire information on the flow of air from the inlet.

2. The inhalation device of claim 1 wherein the sensor and the light signal device are positioned across from each other in the channel such that the vaporized substance can flow between the sensor and the light signal device.

3. The inhalation device of claim 1 wherein the light sensor and the light signal device are positioned next to each other.

4. The inhalation device of claim 1 wherein the light sensor and the light signal device are positioned at an angle in the channel of the inhalation device.

5. The inhalation device of claim 1 further comprising a processor, wherein said processor using data from the light sensor and airflow sensor meters the consumption of the vaporized substance.

6. The inhalation device of claim 1, wherein the airflow sensor comprises an air flow sensor.

7. The inhalation device of claim 1, wherein the airflow sensor comprises a propeller.

8. The inhalation device of claim 1, wherein the airflow sensor comprises a microphone.

9. The inhalation device of claim 1, wherein the airflow sensor comprises a fin.

10. The inhalation device of claim 1, wherein the airflow sensor comprises a heating element

11. The inhalation device of claim 10, wherein the airflow sensor further comprises a temperature sensor.

12. The inhalation device of claim 10, wherein the heating element is configured to have electricity flow through the heating element, and the airflow sensor further comprises a sensor to measure current or resistance in the heating element.

13. An inhalation device for inhaling a vaporized substance comprising:
an inlet;
an outlet;
a channel positioned between the inlet and outlet;
an atomizer positioned between the inlet and the outlet and configured to vaporize an unvaporized substance into a vaporized substance, wherein the vaporized substance flows downstream from the atomizer to the outlet via the channel;
an airflow sensor positioned upstream of the flow of the vaporized substance,
wherein the airflow sensor is configured to acquire information on the flow of air from the inlet.

14. The inhalation device of claim 13, wherein the airflow sensor comprises an air flow sensor.

15. The inhalation device of claim 13, wherein the airflow sensor comprises a propeller.

16. The inhalation device of claim 13, wherein the airflow sensor comprises a microphone.

17. The inhalation device of claim 13, wherein the airflow sensor comprises a fin.

18. The inhalation device of claim 13, wherein the airflow sensor comprises a heating element

19. The inhalation device of claim 18, wherein the airflow sensor further comprises a temperature sensor.

20. The inhalation device of claim 13 further comprising a processor, wherein said processor using data from the airflow sensor meters the consumption of the vaporized substance or determines when a user has begun inhaling.

21. An inhalation device for inhaling a vaporized substance comprising:
a chamber comprising:
an inlet;
a first channel;
an airflow sensor positioned in the first channel;
a vapor sensing unit comprising:
an outlet;
a second channel;
a light signal device, wherein the light signal device emits light;
a light sensor, wherein the light sensor senses the light from the light signal device;
an atomizer configured to vaporize an unvaporized substance into a vaporized substance, the atomizer positioned between the first and second channel;
wherein the light signal device and light sensor are positioned in the second channel such that the vaporized substance can flow from the atomizer and past the sensor and the light signal device to the outlet;
wherein the airflow sensor is configured to acquire information on the flow of air from the inlet; and
wherein the vapor sensing unit and the atomizer are detachably coupled to the chamber.
